(19) United States
(12) Patent Application Publication
(10) Pub. No.: US 2009/0105753 A1
(43) Pub. Date: Apr. 23, 2009

(54) SUTURES AND METHODS OF MAKING THE SAME

(75) Inventors: E. Skott Greenhalgh, Wyndmoor, PA (US); Neal C. Smith, Hamburg, PA (US)

Correspondence Address: LEVINE BAGADE HAN LLP, 2483 EAST BAYSHORE ROAD, SUITE 100, PALO ALTO, CA, 94303, US

(73) Assignee: Prodesco, Inc., Perkasie, PA (US)

(21) Appl. No.: 11/574,222

(22) PCT Filed: Aug. 26, 2005
(86) PCT No.: PCT/US05/30345

§ 371 Date: Mar. 25, 2008

Related U.S. Application Data
(60) Provisional application No. 60/605,047, filed on Aug. 26, 2004.

(51) Int. Cl. A61B 17/04

(52) U.S. Cl. 606/228

(57) ABSTRACT

A non-braided suture and methods of making and using the same are disclosed. The suture can be woven, for example, from warp yarns and a fill yarn. The suture can have a sheath. The sheath can have a lubricious coating on the inside and the outside of the sheath. The suture can have a textured surface. The suture can furcate. Methods of using the suture are also disclosed.

### BACKGROUND OF THE INVENTION

1. Field of the Invention

This invention relates to sutures and methods of making the same.

2. Description of the Related Art

Sutures have been around for a long time, and are used by all medical professionals in many fields including orthopedics, cardiology, general surgery, neurological and neurovascular surgery. Sutures are made from raw materials including metals (e.g., Stainless, MP35N, Vitalium, gold, platinum), polymers (e.g., PET, PP, nylon, PE, PU, PGA, PLLA, PCL, PLA, PGA), collagen, and combinations of materials.

Sutures are either constructed from monofilaments of multifilament yarns. A suture can be either a stand along yarn (typically monofilament) or multiple yarns braided into a bigger structure.

The constituents of a suture (e.g., braid specs, polymer type, size of suture, lubricant used) define the handling properties of a yarn. Handling is defined by strength, flexibility, softness, ability to hold a knot, strength after a knot is applied, surface roughness, ability to slip through tissue, ability to slip a knot (like tying shoelaces), and many more. Most of these are defined and measured in USP codes or ASTM methods.

### BRIEF SUMMARY OF THE INVENTION

A multi-filament suture for biological use is disclosed. The suture has a warp yarn, a fill yarn, and the warp yarn and fill yarns are woven. The suture can have a sheath. The sheath can be woven. The sheath can be made from yarn and/or a solid formed material, such as a polymer or metal. The suture can have a core. The sheath can substantially surround the core. The yarns and/or sheath can elute agents, such as antibiotics and/or therapeutic pharmaceuticals that can aid healing.

A method for making a multi-filament suture for biological use is also disclosed. The suture is woven from at least one fill yarn and at least one warp yarn. The fill yarn can be tensioned. The method can include encompassing a core in a sheath. The method can include adding an agent to the suture, where the agent can elute from the suture.

### BRIEF DESCRIPTION OF THE DRAWINGS

FIG. 1 illustrates an embodiment of the suture.

FIG. 2 illustrates a side longitudinal sectional view of an embodiment of the suture.

FIG. 3 illustrates a top longitudinal sectional view of the suture of FIG. 2.

FIG. 4 illustrates an embodiment of transverse section A-A.

FIG. 5 illustrates an embodiment of the suture.

FIGS. 6-9 illustrate various embodiments of transverse section A-A.

FIG. 10 illustrates a perspective view of an embodiment of the suture.

FIG. 11 illustrates a top view of the embodiment of the suture of FIG. 7.

FIGS. 12-14 illustrate various embodiments of the suture.

FIG. 15 illustrates an embodiment of the suture having various embodiments of barbs.

FIG. 16 illustrates an embodiment of the yarn or suture.

FIG. 17 illustrates an embodiment of the suture.

FIGS. 18 and 19 illustrate a method of forming a weave pattern into the sheath.

FIG. 20 illustrates an embodiment of two sutures in a knot.

FIGS. 21-24 illustrate various methods of deploying the suture.

### DETAILED DESCRIPTION

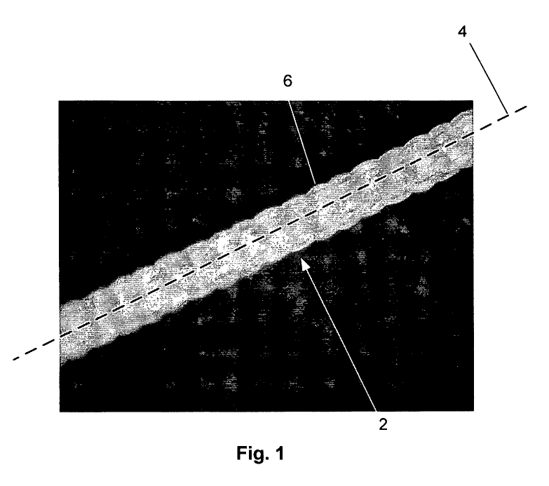

FIG. 1 illustrates a non-braided, woven multi-filament suture 2. The suture 2 can be made from any of the materials listed herein. The suture 2 can have a longitudinal axis 4. The suture 2 can have one or more warp yarns 6. The surface of the suture 2 can have a high friction texture, for example an undulating, corrugated, ridged, grooved, wrinkled, furrowed or otherwise roughened surface, such as shown. The surface of the suture 2 can have a low friction texture, for example by covering the surface with a low friction sheath.

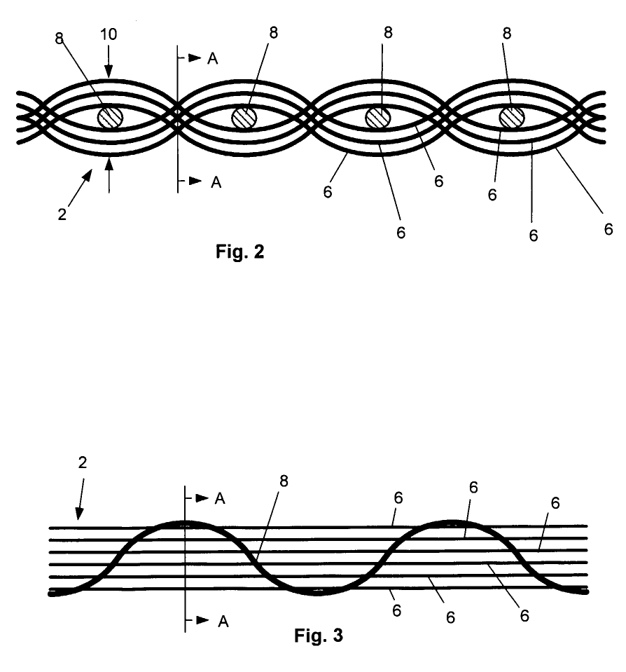

FIGS. 2 and 3 (not to scale) illustrate a longitudinal section of the suture 2. FIG. 2 shows a side view. FIG. 3 shows a top view. Only six warp yarns 6 and one fill yarn 8 are shown for clarity, but the suture 2 can have more or less warp yarns 6 and one or more fill yarns 8. The fill yarns 8 can pass between sets of warp yarns 6 in a regular or irregular periodic configuration, such as sinusoidally as shown in FIG. 3.

The suture 2 can have a suture diameter 10. The suture diameter 10 can be from about 12 USP suture diameter to about 0 USP suture diameter. The suture diameter 10 can be constant relative to the longitudinal distance along the suture 2. The suture diameter 10 can change relative to the longitudinal distance along the suture 2. The change in the suture diameter 10 relative to the longitudinal distance along the suture can be one or more discrete (i.e., substantially instantaneous, immediate) changes and/or one or more lengths of continuous and/or increasing and/or decreasing change. The continuous and/or increasing and/or decreasing change in the suture diameter 10 can be gradual.

The tension in the fill yarns 8 can be adjusted to produce sutures 2 that can have different mechanical characteristics. When the tension in the fill yarns 8 is increased, the suture diameter 10 can decrease and the hardness of the suture 2, and the modulus of elasticity of the suture 2 can increase. The tension in the fill yarns 8 can be constant relative to the longitudinal distance along the suture 2. The tension in the fill yarns 8 can change relative to the longitudinal distance along the suture 2. The change in the tension in the fill yarns 8 relative to the longitudinal distance along the suture can be one or more discrete (i.e., substantially instantaneous, immediate) changes and/or one or more lengths of continuous and/or increasing and/or decreasing change. The continuous and/or increasing and/or decreasing change in the tension in the fill yarns 8 can be gradual.

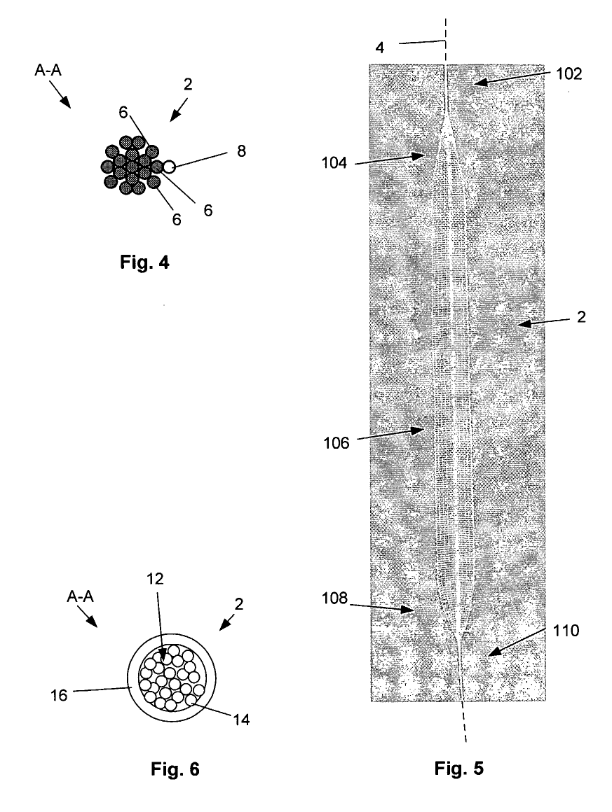

FIG. 4 illustrates that a transverse section of the suture 2 can have a number of warp yarns 6 and one or more fill yarns 8. The transverse section of the suture 2 can have a transverse suture configuration (e.g., shape and/or size). The transverse suture configuration can be substantially round, circular (as shown), oval, square, rectangular, triangular, hexagonal, or combinations thereof. The transverse suture configuration can be substantially flat. The fill yarn 8 can be on the outside of shape formed by the transverse section. The transverse suture configuration can be constant relative to the longitudinal distance along the suture 2. The transverse suture configuration can change relative to the longitudinal distance along the suture 2. The change in the transverse suture configuration relative to the longitudinal distance along the suture can be one or more discrete (i.e., substantially instantaneous, immediate) changes and/or one or more lengths of continuous and/or increasing and/or decreasing change. The continuous and/or increasing and/or decreasing change in the transverse suture configuration can be gradual.

FIG. 5 illustrates that the suture 2 can have one, two, three or more configurations along the longitudinal length of the suture 2. A first length 102 can have a first configuration (e.g., shown as round). A second length 104 can change from the first configuration to a second configuration. A third section 106 can have the second configuration (e.g., shown as flat). A fourth length 108 can change from the second configuration to a third configuration. The second length 104 and/or the fourth length 108 can have substantially no length, for example, when the change between the configurations in the adjacent lengths is discrete. A fifth length 110 can have the third configuration (e.g., shown as round). The first and third configurations can be the same or different configurations.

FIG. 6 illustrates that the suture 2 can have a core 12 surrounded by a sheath 16. The core 12 can have core yarns 14, for example warp yarns 6 and/or fill yarns 8. The sheath 16 can have a solid (as shown), woven, or braided composition. The sheath 16 can be a hollow cylinder, for example a molded or otherwise formed solid piece of a polymer and/or metal. The sheath 16 can be configured to slide against the core 12. The sheath 16 can be fixed to the core 12 at fixation points and/or fixation areas (not shown) along the longitudinal axis 4. The sheath 16 can have different mechanical properties (e.g., hardness and softness, modulus of elasticity, hysteresis loss, yield strength, surface friction, lubricating agents) than the core 12.

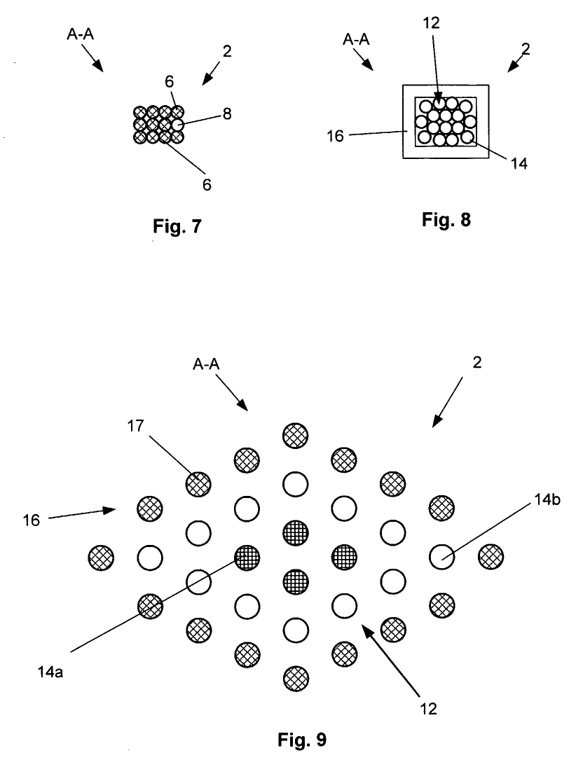

FIG. 7 illustrates that the transverse suture configuration can form a rectangular shape. The fill yarn 8 can be incorporated in the shape formed by the transverse suture configuration (e.g., the rectangle shown in FIG. 7).

FIG. 8 illustrates that the transverse section of the sheath can be a transverse sheath configuration. The transverse sheath configuration can form a rectangular shape. The transverse sheath configuration can be constant relative to the longitudinal distance along the suture 2. The transverse sheath configuration can change relative to the longitudinal distance along the suture 2. The change in the transverse sheath configuration relative to the longitudinal distance along the suture can be one or more discrete (i.e., substantially instantaneous, immediate) changes and/or one or more lengths of continuous and/or increasing and/or decreasing change. The continuous and/or increasing and/or decreasing change in the transverse sheath configuration can be gradual.

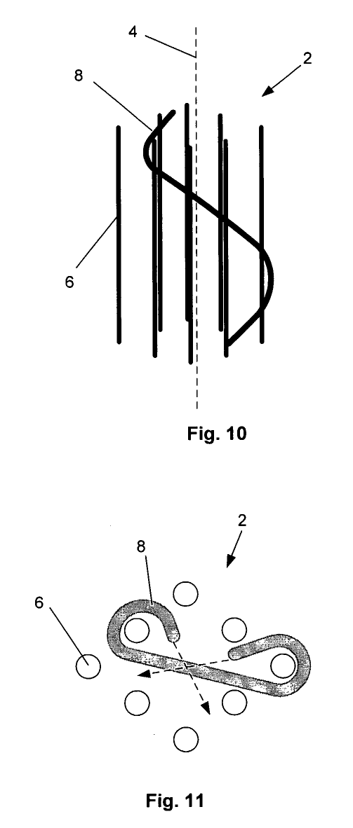

FIG. 9 illustrates that the sheath 16 can have sheath yarns 17. The sheath yarns 17 can be the warp yarns 6 and/or fill yarns 8 as described supra, and/or the sheath yarns 17 can be other yarns. The core 12 can have one or more sets of core yarns. For example, the core 12 can have inner core yarns 14a and outer core yarns 14b. Each set of core yarns 14 can have different mechanical properties from each other set of core yarns 14. Each set of core yarns 14 can have one or more yarns. Each set of core yarns 14 can be interwoven for part or all of the length of the given set of core yarns 14 with one or more sets of core yarns 14 and/or sheath yarns 17.

One or more sets of core yarns 14 and/or sheath yarns 17 can be marker bands. The marker bands can be colored threads, radiopaque threads, yarns and/or systems (e.g., gold, platinum, iridium, rhenium, tantalum, or alloys and/or combinations thereof).

The suture can have one, two, three or more sheaths 16. Each sheath 16 can be on top or below (i.e., overlay) each other, and/or each sheath 16 can be at different longitudinal distances along the suture 2 from the other sheaths 16. Each sheath 16 can be interwoven with one or more other sheath 16 (e.g., by interweaving the sheath yarns 17).

Each set of core yarns 14 can have a core yarn length. Each sheath 16 can have a sheath length. Each sheath yarn 17 can have a sheath yarn length. The core yarn lengths and/or sheath lengths and/or sheath yarn lengths can be part or all of the length of the suture 2.

The suture 2 can have hollow lengths. The hollow length can be a length of the suture 2 that can have the sheath 16 and an absence of core yarns 16. For example, a single sheath 16 can have two or more sets of the core yarns 14 within the sheath 16, where a gap of no core yarns 14 exists in the sheath between two of the sets of core yarns 14. The hollow length can be part or all of the entire length of the suture 2.

The suture 2 can have exposed lengths. The exposed length can be a length of the suture 2 that can have one or more core yarns 16 and an absence of the sheath 16. For example, one or more core yarns 14 can have two or more sheaths 16 on the core yarns 14, where a gap of no sheath 16 exists on the core yarns 14 between two of the sheaths 16. The exposed length can be part or all of the entire length of the suture 2.

FIGS. 10 and 11 illustrate the fill yarn 8 that can be tightened and/or fixed and/or wrapped around warp yarns 6 as the fill yarn 8 passes along the length of the longitudinal axis 4. As shown by the phantom arrows in FIG. 11, the fill yarn 8 can continue to wrap around different warp yarns 6 as the fill yarn 8 proceeds up and down the suture 2 along the longitudinal axis 4.

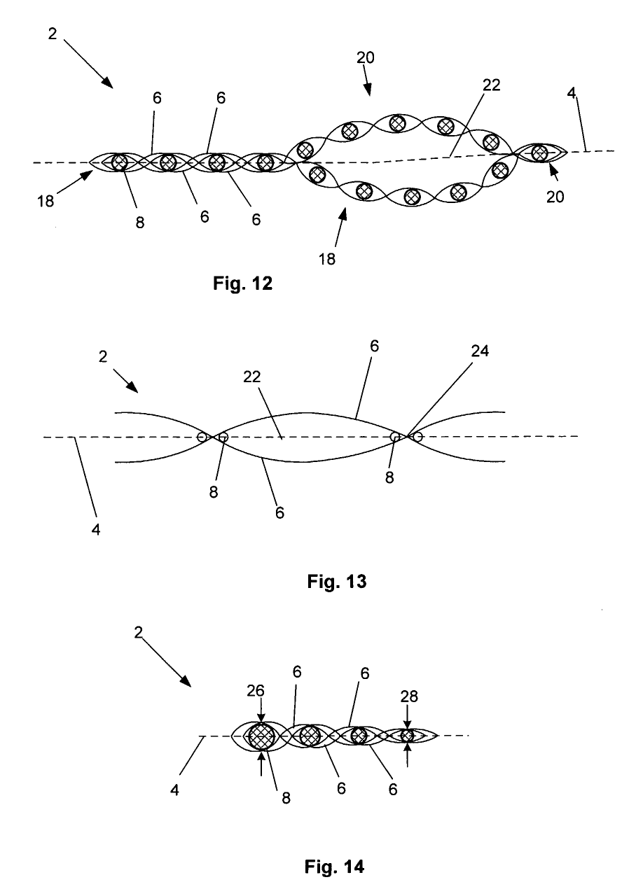

FIG. 12 illustrates that the suture 2 can furcate, for example bifurcate (as shown) or trifurcate. The suture can remain furcated along the remainder of the length of the suture after furcating. The suture 2 can merge after furcating. The suture 2 can have a first composite strand 18. The first composite strand 18 can separate (i.e., furcate) into a first strand 18 and a second strand 20. The first strand 18 and the second strand 20 can each have fill yarns 8 and warp yarns 6. The first strand 18 and the second strand 20 can each be woven. Additional strands (not shown) can be in the first composite strand 18. The first strand 18 and second strand 20 can come together (i.e., merge) to form a second composite strand 20.

A port 22 can be formed between where the first strand 18 separates from the second strand 20 at the first composite strand 18, and where the first strand 18 comes together with the second strand 20 at the second composite strand 20. The port 22 can be used as a buttonhole. An end of the suture 2 or another suture can be pulled through the port 22 to form a knot. Other elements (e.g., elastic elements, needles) can be passed into the port 22 and attached to the suture 2. An object (e.g., another suture, catheter, staple, tube, guidewire, post) can be pulled or inserted through the port 22, and the first composite strand 18 and the second composite strand 20 can be twisted about the longitudinal axis 4 such that the first composite strand 18 and the second composite strand 20 can twist down onto the object, securing the object in the port 22.

FIG. 13 illustrates that the suture 2 can have a warp yarn crossing 24 where the warp yarns 6 substantially intersect. The fill yarns 8 can be placed adjacent to the warp yarn crossings 24 such that the warp yarns 6 can form substantial angles with respect to the longitudinal axis 4. The warp yarns 6 can form ports 22 between the warp yarn crossings 24.

The suture 2 can have a port 22 woven into the suture 2. The port 22 can be woven into the suture 2 as described supra. The port 22 can be woven into the suture 2 by leaving out a length of one or more fill yarns 8 and/or warp yarns 6.

The suture 2 can not come back together after furcating. For example, the suture 2 can have multiple strands extending from a composite strand, where the multiple strands have terminal ends away from the composite strand. The suture 2 can be driven into, for example, a tissue and/or medical device and multiple strands that can have terminal ends can extend from the tissue and/or medical device.

FIG. 14 illustrates that the suture 2 can have a first diameter 26. The suture 2 can have a second diameter 28 at a length along the longitudinal axis 4. The first diameter 26 can be greater than the second diameter 28. The diameter of the suture 2 can gradually taper along the length of the longitudinal axis 4 from the first diameter 26 to the second diameter 28. The diameter of the suture 2 can immediately step from the first diameter 26 to the second diameter 28. The diameter change can be due to a change along the length of the suture 2 in, for example, warp yarn diameter, fill yarn diameter, the number of warp yarns 6, the number of fill yarns 8, the tension of the fill yarns 8, or combinations thereof.

FIG. 15 illustrates that the suture 2 can have one or more retention configurations or devices, for example barbs. A first barb 112 can have one or more of the radially outer warp yarns 6 (as shown) and/or sheath yarns 17. A second barb 114 can have one of more of the radially intermediate and/or inner warp yarns 6.

The first and/or second barbs 112 and/or 114 can be formed by weaving the suture 2 and then cutting one or more of the appropriate yarns (e.g., the specific core yarns 14 and/or sheath yarns 17). The first and/or second barbs 112 and/or 114 can be formed by making the suture 2 with multiple appropriate yarns of the correct length to terminate at the first and/or second barb 112 and/or 114.

A third barb 116 can be a yarn and/or a second suture, and/or a shaft of metal and/or polymer. The third barb 116 can be substantially straight. The third barb 116 can be releasably or fixedly attached (e.g., threaded, woven, adhered such as glued, welded), to the warp yarns 6 and/or the fill yarns 8. The third barb 116 can pass through a sheath 16 and/or be releasably or fixedly attached to the sheath yarns 17.

A fourth barb 118 can be curved. The fourth barb 118 can be releasably or fixedly attached to one or more warp yarns 6.

A fifth barb 120 can be curved. The curve of the fifth barb 120 can be, for example, from about 180 degrees to about 360 degrees, for example, about 345 degrees. about or more than about degrees. The fifth barb 120 can be releasably or fixedly attached to one or more warp yarns 6. The fifth barb 120 can be releasably or fixedly attached to the fill yarn 8. The fifth barb 120 can be configured to pass around the fill yarn 8, for example, so that the fill yarn 8 can retain the fifth barb 120 during use.

A sixth barb 122 can be curved. The curve of the sixth barb 122 can be, for example, about or more than about 360 degrees, for example about 540 degrees. The sixth barb 122 can be releasably or fixedly attached to one or more warp yarns 6. The sixth barb 122 can be releasably or fixedly attached to the fill yarn 8. The sixth barb 122 can be configured to pass around the fill yarn 8, for example, so that the fill yarn 8 can retain the sixth barb 122 during use.

The suture 2 can have any combination or single embodiment of barbs. The suture can have any number of barbs.

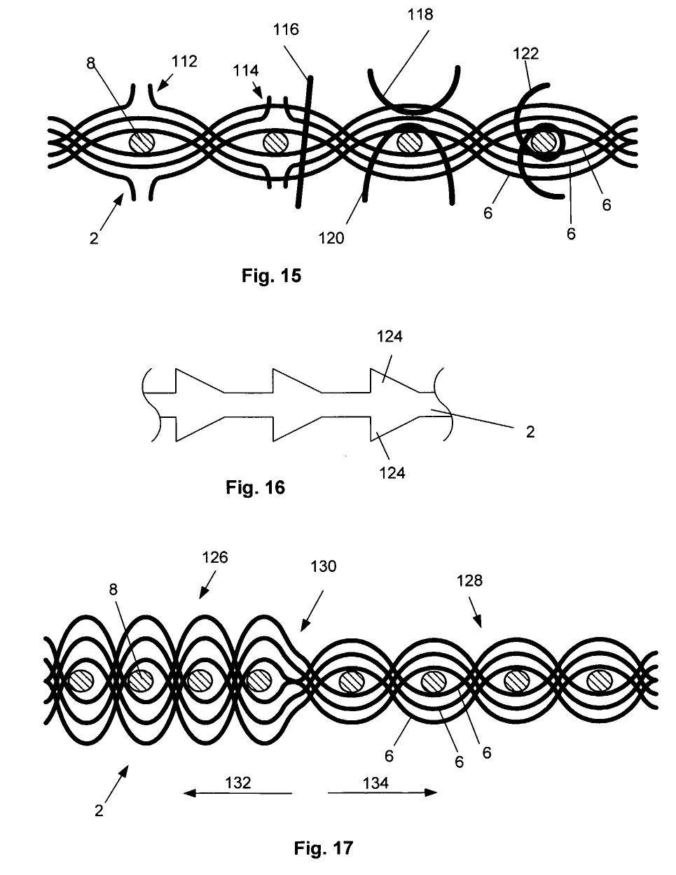

FIG. 16 illustrates the suture 2 or yarn (not shown) that can have a retaining configuration 124. The retaining configuration 124 can be configured to produce high frictional and/or interference forces when a force in the first direction is applied to the suture 2 or yarn, and low frictional and/or interference forces when a force in the second direction is applied to the suture 2 or yarn. The retaining configuration 124 can, for example, be a sharktooth configuration, a wedge, a barb, a pin, a cone, a directional knurled surface texture, another directional surface texture, or combinations thereof.

FIG. 17 illustrates the suture 2 that can have a first textured length 126 and a second textured length 128. The fill yarn 8 in the first textured length 126 can be at a first tension. The fill yarn 8 in the second textured length 128 can be at a second tension. The first tension can be greater than the second tension. The first textured length 126 can have a high friction surface. The second textured length 128 can have a low friction surface. A directional texture length 130 can be a high friction texture when the suture 2 is translated in a first direction 132. The directional texture length 130 can be a low friction texture when the suture 2 is translated in a second direction 134. The directional configuration (e.g., the lower slope on the side of the second direction, and the higher slope on the side of the first directional) can be formed, for example, from the fill yarn 8 transitioning from a high tension to a low tension.

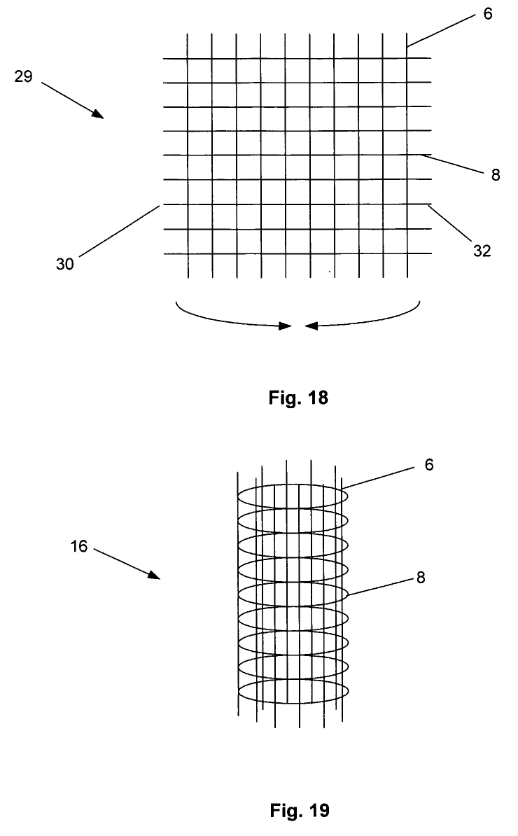

FIG. 18 illustrates a weave pattern 29 that can be made from warp yarns 6 and fill yarns 8, possibly, but not necessarily the same warp yarns 6 and fill yarns 8 described supra to make the suture 2. The fill yarns 8 can have first fill ends 30 and second fill ends 32. The weave pattern 29 can be substantially flat and/or two-dimensional. As shown by the arrows, the first fill ends 30 and the second fill ends 32 can be brought together. The first fill ends 30 can then be fixedly attached to the second fill ends 32.

FIG. 19 illustrates that the weave pattern 29 of FIG. 18 can form the sheath 16, for example after the first fill ends 30 have been brought together with, and fixedly attached to, the second fill ends 32. The sheath 16 can be substantially cylindrical. The weave pattern 29 can change from a first weave pattern to a second weave pattern along the longitudinal length and/or along the angle of the sheath 16 and/or the suture 2.

Any or all elements of the suture 2 and/or other devices or apparatuses described herein can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), nickel-cobalt alloys (e.g., MP35N® from Magellan Industrial Trading Company, Inc., Westport, Conn.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as ultra-high molecular weight polyethylenes (e.g., DYNEEMA® from DSM, Heerlen, The Netherlands; SPECTRA® from Honeywell International, Inc., Morristown, N.J.; VECTRAN® from Celanese AG, Kroberg im Taunus, Germany) polyethylene teraphathalate (PET), polyester (e.g., DACRON® from E. I. Du Pont de Nemours and Company, Wilmington, Del.), polypropylene, (PET), polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), absorbable or resorbable polymers such as polyglycolic acid (PGA), polylactic acid (PLA), polycaprolactone (PCL), polyethyl acrylate (PEA), polydioxanone (PDS), and pseudo-polyamino tyrosine-based acids, extruded collagen, silicone, zinc, echogenic, radioactive, radiopaque materials, any of the other materials listed herein or combinations thereof. Examples of radiopaque materials are barium sulfate, zinc oxide, titanium, stainless steel, nickel-titanium alloys, tantalum and gold.

Any or all elements of the suture 2 and/or other devices or apparatuses described herein, can be or have a matrix for cell ingrowth or used with a fabric, for example a covering (not shown) that acts as a matrix for cell ingrowth. The matrix and/or fabric can be, for example, polyester (e.g., DACRON® from E. I. Du Pont de Nemours and Company, Wilmington, Del.), polypropylene, PTFE, ePTFE, nylon, extruded collagen, silicone or combinations thereof.

The elements of the suture 2 and/or other devices or apparatuses described herein and/or the fabric can be filled and/or coated with an agent delivery matrix known to one having ordinary skill in the art and/or a therapeutic and/or diagnostic agent. The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

The suture 2 can be made from materials that can bioabsorb or otherwise erode, as known to those having ordinary skill in the art. The eroding sections can be specific yarns, lengths, diameters, depths, and/or surfaces of the suture 2. The suture 2 can deliver one or more agents at specific yarns, lengths, diameters, depths, and/or surfaces of the suture 2. The elasticity of the material of the suture 2 can change for specific yarns, lengths, diameters, depths, and/or surfaces of the suture 2.

The materials used to make the warp yarns 6 and/or the fill yarns 8 and/or the sheath 16 and/or any other element of the suture 2 or the suture 2 as a whole can be constant relative to the longitudinal distance along the suture 2. The materials used to make the warp yarns 6 and/or the fill yarns 8 and/or the sheath 16 and/or any other element of the suture 2 or the suture 2 as a whole can change relative to the longitudinal distance along the suture 2. The change in the materials used to make the warp yarns 6 and/or the fill yarns 8 and/or the sheath 16 and/or any other element of the suture 2 or the suture 2 as a whole relative to the longitudinal distance along the suture can be one or more discrete (i.e., substantially instantaneous, immediate) changes and/or one or more lengths of continuous and/or increasing and/or decreasing change. The continuous and/or increasing and/or decreasing change in the materials used to make the warp yarns 6 and/or the fill yarns 8 and/or the sheath 16 and/or any other element of the suture 2 or the suture 2 as a whole can be gradual.

FIG. 5 can illustrate that the suture 2 can have one, two, three or more material compositions along the longitudinal length of the suture 2. The first length 102 can have a first material composition. The second length 104 can change from the first material composition to a second material composition. The third section 106 can have the second material composition. The fourth length 108 can change from the second material composition to a third material composition. The second length 104 and/or the fourth length 108 can have substantially no length, for example, when the change between the material composition in the adjacent lengths is discrete. The fifth length 110 can have the third material composition. The first and third material compositions can be the same or different material compositions. For example, the first and third material compositions can be substantially 100% PET. For example, the second material composition can be substantially 100% PEG. The material compositions can change concurrent (as described supra) and/or not concurrent with changes in the configurations.

The suture 2 can have additional elements, for example, metallic grommets, rivets, anchors, tubes, pins, spheres, combinations thereof. The additional elements can be woven into the suture 2.

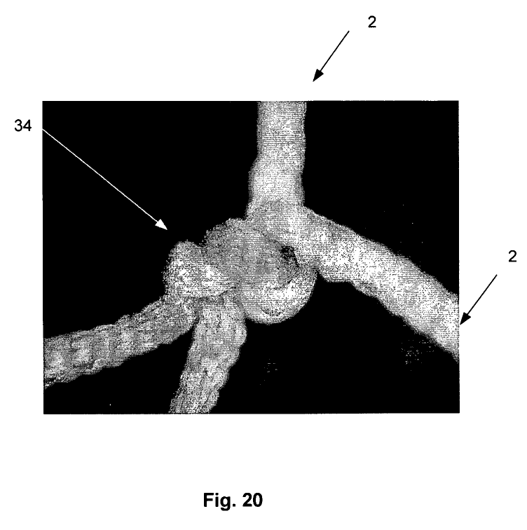

FIG. 20 illustrates a method of using two sutures 2 to form a knot 34. The textures of the surface of the sutures 2 can assist in retaining the knot 34.

The suture 2 can have different elements effecting the performance characteristics of the suture 2. Adjusting the type of material (e.g., polymer, metal), number of yarn ends and/or size of the yarn can effect the strength of the suture 2. Adjusting the material type (e.g., polymer) and/or density of the weave or yarns (i.e., a tighter, high density weave is smoother), can effect the knot slip of the suture 2. Adjusting the material type, density of weave or yarns, and/or if a polymer resides within the woven structure, where the polymer resides within the woven suture, can effect the knot strength of the suture 2. Adjusting the polymer type (e.g., mixing a soft polymer within the suture 2), and/or suture surface roughness, can effect knot retention of the suture 2. Weaving in a mix of various biodegrading polymers and/or changing the porosity of the suture can effect the long-term strength of the suture 2. The weave or yarn density and/or tension in the fill yarns 8 or warp yarns 6 can have an effect on stiffness in the suture 2. The polymer type, the crimp of the weave (i.e., high crimp is springier), the use of wrapped yarns (e.g., PET around Polyurethane), and/or the use of a partially oriented yarn (POY) can effect the elongation and/or elasticity of the suture 2.

The suture 2 can be configured to deform (e.g., dent) under pressure over the length of the suture 2 where the suture 2 is tied in a knot or otherwise frictionally restrained, for example, to improve knot retention. The core yarns 14 and/or sheath yarns 17 can be POY along the entire length of the core yarns 14 and/or sheath yarns 17, and/or along the length of the core yarns 14 and/or sheath yarns 17 where the suture 2 is tied in a knot or otherwise frictionally restrained. The suture 2 or elements thereof can be made from softer materials and/or a softer configuration (e.g., more porous, less dense weave) along the length of the suture 2 where the suture 2 is tied in a knot or otherwise frictionally restrained, compared to the materials and/or configuration for the remainder of the suture 2 or elements thereof. The suture 2 or elements thereof can have higher friction surface texturing along the length of the suture 2 where the suture 2 is tied in a knot or otherwise frictionally restrained, compared to the surface texturing for the remainder of the suture 2 or elements thereof.

FIG. 21 illustrates that the suture 2 can be pushed or pulled, as shown by arrow, through tissue 136. A needle 138 can be fixedly or removably attached to the end of the suture 2. The suture 2 can have the core 12 and the sheath 16. The suture 2 can have an outer sheath (represented by sheath 16 in FIGS. 21 through 24 and in the text infra) over an inner sheath (represented as part of the core 14 in FIGS. 21 through 24 and the text infra).

The sheath 16 can be fixedly and/or releasably and/or slidably attached to the core 12 along all or part of the length of the suture 2. The sheath 16 can be fixedly and/or removably attach to the core 12 at attachment area 140. The attachment area 140 can be adjacent to the end of the suture 2. The sheath 16 at the attachment area 140 can be the failure zone for tensile stress on the sheath 16. The sheath 16 at the attachment area 140 can be perforated.

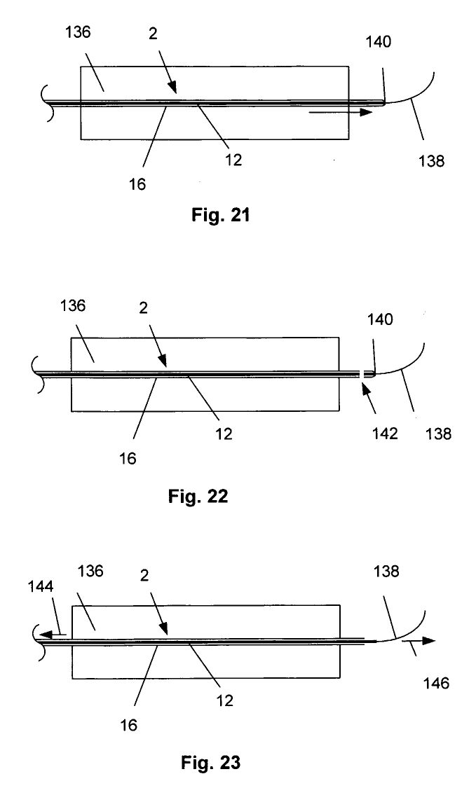

FIG. 22 illustrates that the suture 2 can be split (e.g., cut) substantially perpendicular to the longitudinal axis (not shown in FIG. 22). The suture 2 can be split between the needle 138 and the tissue 136. When the suture 2 is split, the sheath 16 can be slidably attached to the core 12.

FIG. 23 illustrates that a first force, shown by arrow 144, can be applied to the sheath 16. A second force, shown by arrow 146, can be applied to the core 12, for example through the needle 138. The first force and the second force can be in opposite directions along the longitudinal axis 4 of the suture 2. The sheath 16 can separate from the core 12, for example at the attachment area 140.

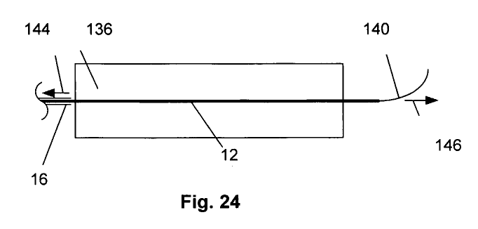

FIG. 24 illustrates that the sheath 16 can be removed from the core 12. The sheath can be retracted from the tissue 136, the core 12 can be exposed to the tissue 136. The exterior surface of the core 12 can be higher friction than the exterior surface of the sheath 16.

The exterior and/or interior of the sheath 16 can be low friction surfaces. The sheath 16 and/or a coating on the interior and/or exterior of the sheath 16 can be dissolvable. The exterior and/or interior and/or the entirety of the sheath 16 can have and/or be a coating. The coating can be an anti-microbial coating. The coating can be a lubricious coating. The lubricious coating can have or be a hydrogel. The coating can have or be any of the materials and/or agents described herein.

As disclosed herein, the transverse shape of the suture 2 can be adjusted (e.g., round to flat, flat to round). Adjusting the transverse sectional shape can optimize stress distributions, and reduce the maximum stresses on yarns in the suture 2. The transverse sectional shape can be adjusted without substantively affecting the strength of the suture 2.

The suture 2 can have a rough, open surface. The rough, open surface can promote tissue in-growth into the suture 2. If the suture 2 is directly molded into a part (e.g., injection molded), the suture 2 can have a bumpy surface to improve the bond strength between the part and the suture 2.

The yarn tension, number of yarns, position of the yarns, weave angle, size of the yarn, polymer type, number of the yarns running in the weave, mixing of yarn layers (e.g., changing through cross section binding or thru weaving systems), mixing of sheath and core, individual yarn tensions, sheath density versus core yarn density, core yarn density, changing density down the suture length, changing diameter down the suture length, and cross section shape down the suture length (e.g., round to flat, and back to round) can be adjusted by the elements and/or use alterations.

The suture 2 and/or any elements herein can be made from shape memory materials (e.g., Nitinol). Part or all of the suture 2 can be preshaped or otherwise preformed into a preformed suture configuration. During use the suture 2 can be deformed or the configuration of the suture otherwise altered. During use, the suture 2 can resiliently or otherwise return to the preformed suture configuration.

As used herein, configuration can refer to shape and/or size and/or other physical characteristics relating to structure.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

We claim:

1. A multi-filament suture for biological use comprising:
a warp yarn; and
a fill yarn;
wherein the suture comprises a weave.

2. The suture of claim 1, further comprising a sheath.

3. The suture of claim 2, wherein the sheath comprises a weave.

4. The suture of claim 2, wherein the sheath comprises yarn.

5. The suture of claim 2, wherein the sheath is a non-yarn material.

6. The suture of claim 2, further comprising a core.

7. The suture of claim 4, wherein the sheath substantially surrounds the core.

8. The suture of claim 6, wherein the core has core mechanical properties, and wherein the sheath has sheath mechanical properties, and wherein the core mechanical properties are not substantially equivalent to the sheath mechanical properties.

9. The suture of claim 1, wherein the suture has a textured surface.

10. The suture of claim 9, wherein the textured surface has a high-friction surface.

11. The suture of claim 10, wherein the textured surface comprises the warp yarns.

12. The suture of claim 10, wherein the textured surface comprises a sheath.

13. The suture of claim 9, wherein the textured surface has a low-friction surface comprising the warp yarns.

14. The suture of claim 1, further comprising an agent, and wherein the suture is configured to elute the agent.

15. A suture for biological use, comprising
A first length having a first end,
A second length having a first end and a second end, wherein the second length comprises multiple strands.
wherein the first end of the first length is integral with the second end of the second length.

16. The suture of claim 15, wherein the first length consists of a single strand.

17. The suture of claim 15, further comprising a third length having a second end, wherein the second end of the third length is integral with the first end of the second length.

18. The suture of claim 15, wherein a first strand in the second length has a first terminal end.

19. The suture of claim 18, wherein a second strand in the second length has a second terminal end.

20. The suture of claim 15, wherein the suture comprises a fill yarn and a warp yarn, and wherein the fill yarn is woven with the warp yarn.

21. A method of making a multi-filament suture for biological use, comprising:
weaving the suture, and wherein a fill yarn is woven into at least one warp yarn.

22. The method of claim 21, further comprising weaving a sheath.

23. The method of claim 22, further comprising encompassing a core in a sheath.
